(19) United States
(12) Patent Application Publication
(10) Pub. No.: US 2012/0179238 A1
(43) Pub. Date: Jul. 12, 2012

(54) STENT HAVING VARIABLE STIFFNESS

(75) Inventors: Timur P. Sarac, Orange Village, OH (US); Craig Bonsignore, Pleasanton, CA (US); Albrecht H. Kramer, Santiago (CL)

(73) Assignees: PeriTec Biosciences, Ltd.; The Cleveland Clinic Foundation

(21) Appl. No.: 12/987,710

(22) Filed: Jan. 10, 2011

(51) Int. Cl. A61F 2/82; B26D 1/00

(52) U.S. Cl. 623/1.16; 623/1.15; 83/13

(57) ABSTRACT

A plurality of radially expandable cylindrical elements are generally aligned along a common longitudinal axis and are interconnected by a plurality of interconnecting members placed so that the stent is flexible in the longitudinal direction. The plurality of cylindrical elements collectively form first and second stent ends longitudinally separated by a stent body. At least one of the first and second stent ends is reverse-tapered laterally outward from the longitudinal axis and longitudinally away from the stent body. The stent body has a stiffness value of X, and at least one of the first and second stent ends has a stiffness value of Z, with Z being greater than

X such that the stent is more resistant to lateral force in the at least one of the first and second stent ends than in the stent body.

### TECHNICAL FIELD

The present invention relates to an apparatus and method for use of a stent and, more particularly, to a stent having variable stiffness along a length thereof.

### BACKGROUND OF THE INVENTION

Expandable endoprosthesis devices, generally known as stents, are designed for implantation in a patient's body lumen (such as a blood vessel) to maintain the patency thereof. These devices are particularly useful in the treatment and repair of blood vessels after a stenosis has been compressed by percutaneous transluminal coronary angioplasty (PTCA), percutaneous transluminal angioplasty (PTA), or removed by atherectomy or other means.

Stents are generally cylindrically-shaped devices which function to hold open and sometimes expand a segment of a blood vessel or other lumen, such as a coronary artery. They are particularly suitable for use to support the lumen or hold back a dissected arterial lining which can occlude the fluid passageway therethrough.

A variety of devices are known in the art for use as stents and include coiled wires in a variety of patterns that are expanded after being placed intraluminally on a balloon catheter; helically wound coiled springs manufactured from an expandable heat sensitive metal; and self-expanding stents inserted in a compressed state and shaped in a zigzag pattern. One of the difficulties encountered using prior art stents involved maintaining the radial rigidity needed to hold open a body lumen while at the same time maintaining the longitudinal flexibility of the stent to facilitate its delivery and accommodate the often tortuous path of the body lumen.

Another problem area has been the limited range of expandability. Certain prior art stents expand only to a limited degree due to the uneven stresses created upon the stents during radial expansion. This necessitates providing stents with a variety of diameters, thus increasing the cost of manufacture. Additionally, having a stent with a wider range of expandability allows the physician to redilate the stent if desired.

Various means have been described to deliver and implant stents. One method frequently described for delivering a stent to a desired intraluminal location includes mounting the expandable stent on an expandable member (such as a balloon) provided on the distal end of an intravascular catheter, advancing the catheter to the desired location within the patient's body lumen, inflating the balloon on the catheter to expand the stent into a permanent expanded condition and then deflating the balloon and removing the catheter. Another known method uses a self-expanding stent which is made of a shape-memory material such as Nitinol™. The self-expanding stent is compressed for insertion into the body, then released within the body and self-expands out to the original size.

It may also be desirable for a stent to have variable strength, yet maintain flexibility so that it can be readily advanced through tortuous passageways and radially expanded over a wider range of diameters with minimal longitudinal contraction to accommodate a greater range of vessel diameters. The expanded stent should have adequate structural strength (hoop strength) to hold open the body lumen in which it is expanded. The control of stent strength at specific locations along the stent may be used to provide a customizable device specifically adapted to the unique body lumen formation in the patient.

### SUMMARY OF THE INVENTION

In an embodiment of the present invention, a stent is disclosed. A plurality of radially expandable cylindrical elements are generally aligned along a common longitudinal axis and are interconnected by a plurality of interconnecting members placed so that the stent is flexible in the longitudinal direction. The plurality of cylindrical elements collectively form first and second stent ends longitudinally separated by a stent body. At least one of the first and second stent ends is reverse-tapered laterally outward from the longitudinal axis and longitudinally away from the stent body. The stent body has a stiffness value of X, and at least one of the first and second stent ends has a stiffness value of Z, with Z being greater than X such that the stent is more resistant to lateral force in the at least one of the first and second stent ends than in the stent body.

In an embodiment of the present invention, a method of forming a stent having a longitudinal axis extending down a stent inner lumen is disclosed. A tubular stent blank laterally enclosing the stent inner lumen and having longitudinally spaced open first and second blank ends separated by a blank body is provided. At least one of the first and second blank ends is reverse-tapered laterally outward from the longitudinal axis and longitudinally outward from the blank body. A plurality of apertures are cut in the stent blank to leave behind a plurality of interconnected struts forming the stent. The stent has first and second stent ends longitudinally separated by a stent body. At least one of the struts is a straight strut, extending substantially parallel to the longitudinal axis. A plurality of struts forming the at least one reverse-tapered stent end each has a selected dimension that has a predetermined relationship to a corresponding selected dimension of each of a plurality of struts forming the stent body. The predetermined relationship is configured to make the stent more resistant to lateral force in the at least one reverse-tapered stent end than in the stent body.

In an embodiment of the present invention, a stent is disclosed. A plurality of struts are interconnected to form a stent having proximal and distal stent ends longitudinally separated by a stent body. A stent inner lumen is laterally enclosed by the proximal and distal stent ends and the stent body. At least one of the proximal and distal stent ends is reverse-tapered laterally outward from the stent body. At least one of the struts is a straight strut, extending substantially parallel to the longitudinal axis. At least one of the struts is an angled strut, extending parallel to a helix centered about the longitudinal axis. A plurality of angled struts are interconnected end-to-end in a zigzag configuration to form a radially expandable cylindrical element extending circumferentially around the stent inner lumen and having a plurality of proximally oriented peaks and distally oriented valleys. Each of the proximal and distal stent ends and the stent body is formed by at least one cylindrical element. A plurality of cylindrical elements are generally aligned along a common longitudinal axis and are interconnected by a plurality of interconnecting members with the proximally oriented peaks of one cylindrical element being located laterally inside the distally oriented valleys of an adjacent cylindrical element. A plurality of substantially longitudinally oriented bridge beams interconnect adjacent cylindrical elements. A plurality of struts forming the at least one reverse-tapered stent end each have a selected dimension that has a predetermined relationship to a corresponding selected dimension of each of a plurality of struts forming the stent body. The predetermined relationship is configured to make the stent more resistant to lateral force in the at least one reverse-tapered stent end than in the stent body.

In an embodiment of the present invention, a stent is disclosed. A plurality of radially expandable cylindrical elements are generally aligned along a common longitudinal axis and are interconnected by a plurality of interconnecting members placed so that the stent is flexible in the longitudinal direction. The plurality of cylindrical elements collectively form first and second stent ends longitudinally separated by a stent body. The stent body has a stiffness value of X, and at least one of the first and second stent ends has a stiffness value of Z, with Z being greater than X such that the stent is more resistant to lateral force in the at least one of the first and second stent ends than in the stent body.

### BRIEF DESCRIPTION OF THE DRAWINGS

For a better understanding of the invention, reference may be made to the accompanying drawings, in which:

FIG. 1 is a side view of one embodiment of the present invention;

FIG. 2 is a front view of the embodiment of FIG. 1;

FIG. 3 is a partial side view of the embodiment of FIG. 1; and

FIG. 4 is a partial side view of the embodiment of FIG. 1.

### DESCRIPTION OF EMBODIMENTS

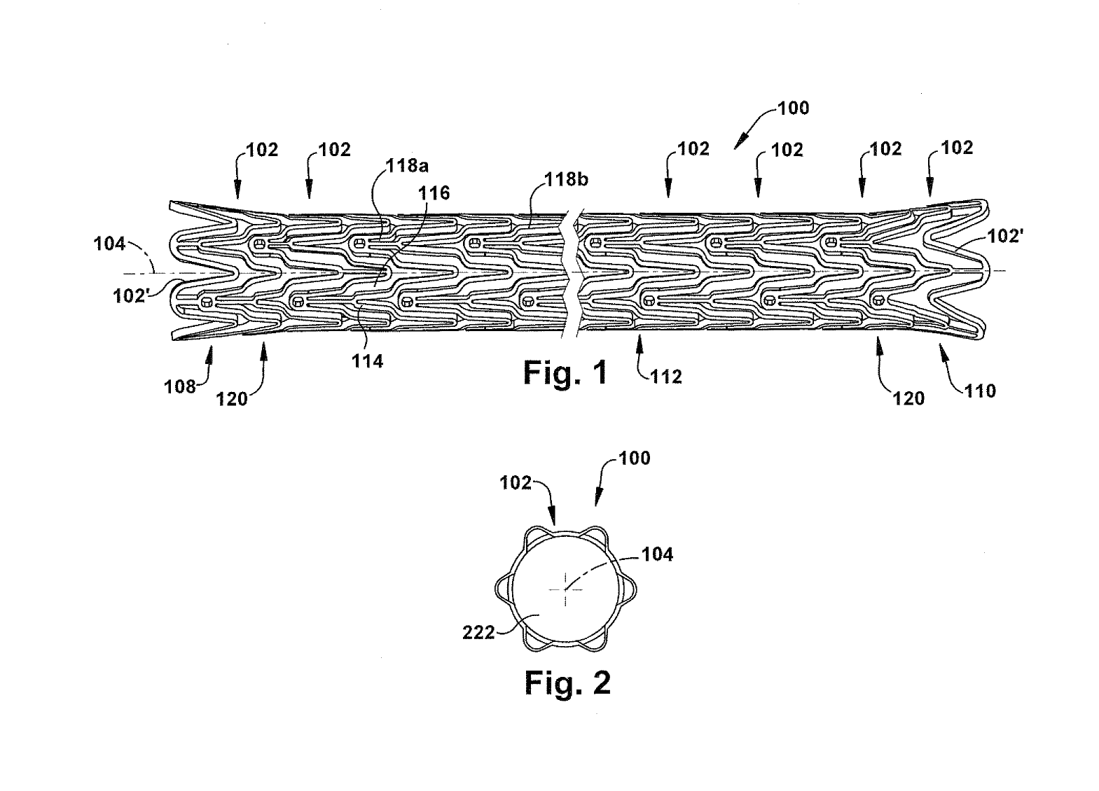

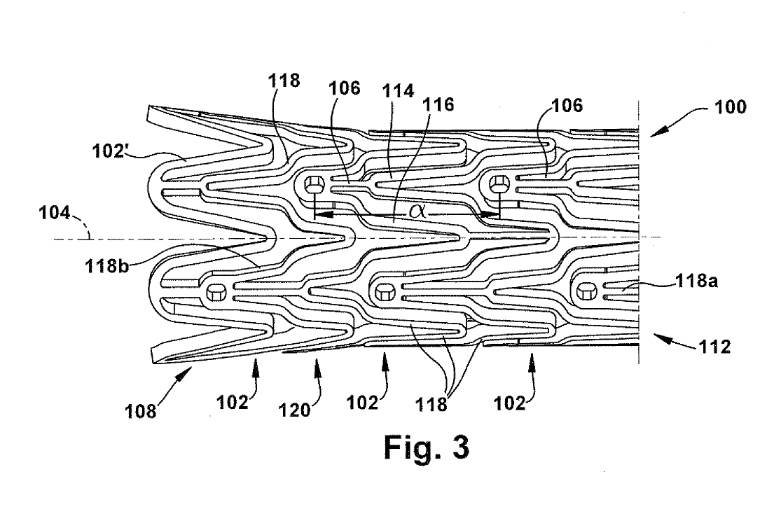

In accordance with the present invention, FIG. 1 depicts a stent 100. A plurality of radially expandable cylindrical elements 102 are generally aligned along a common longitudinal axis 104. The phrase “generally aligned” admits of some degree of mutual offset or obliqueness between the cylindrical elements 102, however. The “longitudinal” direction lies in the plane of the page in the orientation of FIG. 1, as shown. The cylindrical elements 102 (a subset of which are labeled in the Figures, for clarity) are interconnected by a plurality of interconnecting members 106 placed so that the stent is flexible in the longitudinal direction.

The plurality of cylindrical elements 102 collectively form first (proximal) and second (distal) stent ends 108 and 110, respectively, longitudinally separated by a stent body 112. Optionally, and as shown in the Figures, the cylindrical elements 102 of any portion of the stent 100 may each have an alternating-angled or “zigzag” configuration wherein the proximally oriented peaks 114 of one cylindrical element are located laterally inside (e.g., “nested” with) the distally oriented valleys 116 of an adjacent cylindrical element. The “lateral” direction lies in the plane of the page in the orientation of FIG. 2, as shown. Also as shown in the Figures, a plurality of longitudinally oriented bridge beams 106 may serve as the interconnecting members 106, interconnecting adjacent cylindrical elements.

The proximal-most and distal-most cylindrical elements 102′ of the stent 100 may have a different configuration than that of the adjacent cylindrical elements 102, as shown, to provide a “finished” termination to the extreme outer ends of the stent.

Each of the cylindrical elements 102 may include a plurality of struts 118 interconnected in an angular manner to provide a substantially zigzag aspect to the cylindrical element, as will be discussed below. However, each of the individual struts 118, like all structures of the present invention, may have any desired configuration (e.g., the wavelike configuration of the struts shown in the Figures).

At least one of the first and second stent ends 108 and 110 may reverse-taper laterally outward from the longitudinal axis and longitudinally away from the stent body 112. The term “reverse-taper” is used herein to indicate that at least one of the first and second stent ends 108 and 110 flares outward, expanding in diameter as it progresses away from the stent body 112. In contrast, a “tapered” structure would narrow inward by gradually decreasing in diameter from the stent body 112 toward the first or second stent end 108 or 110.

As shown in FIG. 1, both of the first and second stent ends 108 and 110 reverse-taper outward from the stent body 112. Additionally, at least one cylindrical element 102 may form a transition stent portion 120 longitudinally interposed between a chosen one of the first and second stent ends 108 and 110 and the stent body 112. When present, the transition stent portion 120 will normally exhibit physical traits intermediate those that differ between the stent body 112 and either the first or second stent end 108 or 110, whichever is closer to the transition stent portion 120 under discussion.

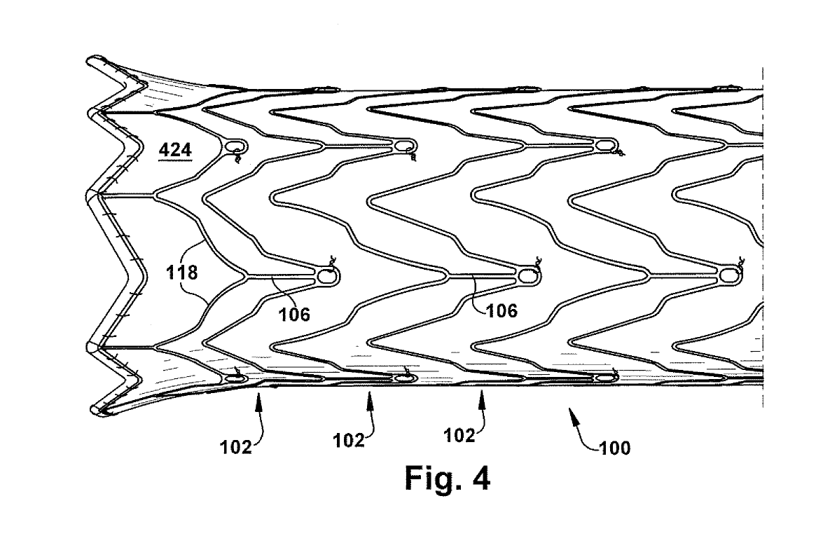

As shown in FIG. 2, the stent 100 may define a stent inner lumen 222 laterally between the cylindrical elements 102 and the longitudinal axis 104. The stent 100 may include a flexible lining tissue 424, visible in FIG. 4, attached to at least one of the cylindrical elements 102 and substantially lining the stent inner lumen 222. The lining tissue 424 may be any suitable nature or artificial tissue, or other substance, having any desired properties for a particular application of the present invention. For example, the lining tissue 424 may be a biological material harvested from any source (such as, but not limited to, bovine, ovine, human, horse, and porcine) including, but not limited to, pericardial tissue, pleural tissue, or peritoneal tissue. As another example, the biocompatible material may also be any suitable synthetic material including, but not limited to, polyurethane or expanded PTFE. It is contemplated that the natural or artificial tissue, substance, or other material will be noticeably thinner in one dimension and will, for some embodiments of the present invention, be a flexible sheet, mesh, web, or other piece of material.

As previously mentioned with reference to FIGS. 1 and 2, the stent 100 may comprise a plurality of struts 118. At least one of the struts 118 may be a straight strut 118a, extending substantially parallel to the longitudinal axis 104. As shown, at least some of the straight struts 118a may be bridge beams 106, interconnecting adjacent cylindrical elements 102. At least one of the struts 118 may also or instead be an angled strut 118b, extending parallel to a helix centered about the longitudinal axis 104. When present, the angled struts 118b may be interconnected end-to-end in a zigzag pattern, as shown in the Figures, to form a cylindrical element 102. Whether or not formed of respective pluralities of struts 118, however, the stent body 112, at least one of the first and second stent ends 108 and 110, and any transition stent portions 120 present may have differing physical properties to provide predetermined characteristics to the stent 100.

More specifically, the stent body 112 may have a stiffness value of X, and at least one of the first and second stent ends 108 and 110 has a stiffness value of 2, where Z is greater than X in some embodiments of the present invention such that the stent 100 is more resistant to lateral force in the at least one of the first and second stent ends than in the stent body for those embodiments of the present invention. When present, a transition stent portion 120 may have a stiffness value of Y, where Y is greater than X and either equal to or less than Z in some embodiments of the present invention, so that the stent 100 may be more resistant to lateral force in the transition stent portion than in the stent body 112 and either the same or less than, respectively, resistant to lateral force in the transition stent portion than in the stent end 108 or 110 closest to the transition stent portion. It should be noted that X, Y (when present, hereafter presumed), and Z do not represent specific absolute values of any particular physical property. X, Y, and Z may have any suitable directly or indirectly proportional relationships to each other. Herein, “stiffness” indicates a lack of flexibility or suppleness.

The relative stiffnesses represented by X, Y, and Z may be achieved in any desired manner for a particular application of the present invention. For example, the stiffness of a particular structure of the stent 100 (e.g., the first stent end 108, second stent end 110, transition stent portion 120, and/or stent body 112) may be directly proportional to the length, or any other dimension, of the struts 118 in that structure and may also be directly proportional to the strain in that structure.

The relative stiffnesses X, Y, and Z of the first and second stent ends 108 and 110, transition stent portion 120, and stent body 112, respectively, may be achieved in any suitable manner. For example, these structures could be made of different materials, subjected to different post-manufacture treatments, include weakened or strengthened portions, or be physically differentiated in any other suitable manner. It is contemplated, however, that the different relative stiffnesses X, Y, and Z will be provided by a relatively uncomplicated dimensional variance between the struts 118 of the first and second stent ends 108 and 110, transition stent portion 120, and stent body 112, respectively. In other words, at least one selected dimension—length along the longitudinal axis 104, width around the circumference of the stent 100, and/or thickness lateral to the longitudinal axis—may have a first value for one of the first and second stent ends 108 and 110, transition stent portion 120, or stent body 112 to provide X, Y, or Z stiffness, and may have a second, different value for another of the first and second stent ends 108 and 110, transition stent portion 120, or stent body 112 to provide X, Y, or Z stiffness. Accordingly, the ratio of a selected dimension of a plurality of struts 118 forming at least one of the first and second stent ends 108 and 110 to that of a corresponding selected dimension of a plurality of struts forming the transition stent portion 120, and to that of a corresponding selected dimension of a plurality of struts forming the stent body 112 might be, for example, 1:A:2A, where A is a chosen number ranging from 1 to 1000, such as, for example, a number in the range of 1 to 50. As an example, the lengths of a plurality of struts 118 forming at least one of the first and second stent ends 108 and 110 might be 2 mm, the lengths of the plurality of struts forming the transition stent portion 120 might be 4 mm, and the lengths of the plurality of struts forming the stent body 112 might be 8 mm. It is contemplated that the selected dimension might not be totally homogenous for each of the struts 118 of the plurality of struts of a selected section (first and/or second stent ends 108 and 110, transition stent portion 120, and/or stent body 112) of the stent 100. However, an average, median, or mean selected dimension, whether mathematically determined, measured, or dead-reckoned by a user, may be sufficient for the purposes of determining the ratios discussed herein.

As an example of suitable stent 100 dimensions for an embodiment of the present invention, the stent may have a total length between 20 and 200 mm, the stent body 112 may have an average expanded diameter between 2 and 50 mm, a plurality of struts 118 forming at least one cylindrical element 112 of at least one of the first and second stent ends 108 and 110 are each between 1 and 500 mm long, a plurality of struts forming a cylindrical element of a transition stent portion 120 interposed longitudinally between a chosen one of the first and second stent ends and the stent body are each between 1 and 500 mm long, and a plurality of struts forming at least one cylindrical element of the stent body are each between 1 and 500 mm long.

With reference to the interconnecting members 106, for certain configurations of the present invention, these interconnecting members may be considered to be intervening bridge beams 106. At least a chosen one of the struts 118 has a selected dimension (length, width, and/or thickness), and at least one of the bridge beams 106 has a corresponding selected dimension (the length, width, and/or thickness that was selected for the strut) that has a value less than the value of the selected dimension of the chosen strut. Therefore, the bridge beam 106 may be more delicate or less robust than the chosen strut 118, due to the different relative selected dimensions.

Similarly, certain of the struts 118 forming the stent 100 may have different relative dimensions. For example, a plurality of struts 118 forming at least a chosen one of the first and second stent ends 108 and 110 may each have a selected dimension (length, width, and/or thickness) that has a predetermined relationship (larger, smaller, or substantially the same value) to a selected dimension of each of a plurality of struts 118 forming the stent body 112. This predetermined relationship may be configured to make the stent 100 more resistant to lateral force (i.e., “stiffer”) in the chosen first or second stent end 108 or 110 than in the stent body 112. This increased stiffness at the first and/or second stent end 108 and 110 from that of the stent body 112 may assist with maintaining flow and/or patency of the body lumen into which the stent 100 is inserted. The increased stiffness may also be helpful in retaining the stent 100 in the desired position within the body lumen, avoiding scarring, and resisting stent fracture. When present, the reverse tapering of the first and/or second stent end 108 and 110 may also, similarly, assist with maintaining patency/flow, retaining the stent 100, avoiding scarring, or resisting stent fracture.

Additionally, when there is at least one transition stent portion 120 longitudinally interposed between the first stent end 108 and the stent body 112 and/or between the second stent end 110 and the stent body 112, the transition stent portion may have physical properties that are intermediate those of the stent body and the first or second stent end 108 or 110 that is closest to that particular transition stent portion. For example, a plurality of struts 118 forming the transition stent portion 120 may each have a selected dimension (length, width, and/or thickness) that has a first predetermined relationship to a corresponding selected dimension (length, width, and/or thickness) of each of the plurality of struts forming the chosen first or second stent end 108 or 110. The plurality of struts 118 forming the transition stent portion 120 may also each have a selected dimension (length, width, and/or thickness) that has a second predetermined relationship to a corresponding selected dimension (length, width, and/or thickness) of each of the plurality of struts forming the stent body 112. The first and second predetermined relationships may chosen to make the stent 100 more resistant to lateral force in the transition stent portion 120 than in the stent body 112 and less resistant to lateral force in the transition stent portion than in at least one of the first and second stent ends 108 and 110.

The stent 100 may be formed in any suitable manner. For example, a tubular stent blank (not shown) which laterally encloses the stent inner lumen 222 may be provided. The stent blank has longitudinally spaced open first and second blank ends separated by a blank body. At least one of the first and second blank ends may be reverse-tapered laterally outward from the blank body. For example, a diverging angle between about 2 and 40 degrees may be imposed between the chosen first or second blank end and the longitudinal axis 104. A plurality of apertures may be cut in the stent blank, before or after the blank ends are reverse-tapered. These apertures may be cut with a laser or any other suitable machine or tool, guided automatically and/or manually. The apertures should be configured and placed to leave behind a plurality of interconnected struts 118 forming the finished stent 100 having first and second stent ends 108 and 110 separated by a stent body 112.

The stent 100 may be made from Nitinol™, stainless steel, nylon, plastic, polymers, or any other material as desired, and may be radiopaque, in whole or part. For ease of description, it is presumed herein that the stent 100 is self-expanding. For example, the struts 118, or any other portions of the stent 100, may be made from a shape memory material, such as, but not limited to, Nitinol™. One of ordinary skill in the art will realize that the stent 100 could instead be expanded using a balloon or other suitable means, and will readily be able to design a deployment system for a stent 100 corresponding to a particular application of the present invention.

While aspects of the present invention have been particularly shown and described with reference to the preferred embodiment above, it will be understood by those of ordinary skill in the art that various additional embodiments may be contemplated without departing from the spirit and scope of the present invention. For example, the specific methods described above for creating and using the stent 100 are merely illustrative; one of ordinary skill in the art could readily determine any number of tools, sequences of steps, or other means/options for placing the above-described apparatus, or components thereof, into positions substantively similar to those shown and described herein. Any of the described structures and components could be integrally formed as a single piece or made up of separate sub-components, with either of these formations involving any suitable stock or bespoke components and/or any suitable material or combinations of materials; however, the chosen material(s) should be biocompatible for most applications of the present invention. Though certain components described herein are shown as having specific geometric shapes, all structures of the present invention may have any suitable shapes, sizes, configurations, relative relationships, cross-sectional areas, or any other physical characteristics as desirable for a particular application of the present invention. Any structures or features described with reference to one embodiment or configuration of the present invention could be provided, singly or in combination with other structures or features, to any other embodiment or configuration, as it would be impractical to describe each of the embodiments and configurations discussed herein as having all of the options discussed with respect to all of the other embodiments and configurations. A device or method incorporating any of these features should be understood to fall under the scope of the present invention as determined based upon the claims below and any equivalents thereof.

Other aspects, objects, and advantages of the present invention can be obtained from a study of the drawings, the disclosure, and the appended claims.

Having described the invention, I claim:

1. A stent, comprising:
a plurality of radially expandable cylindrical elements generally aligned along a common longitudinal axis and interconnected by a plurality of interconnecting members placed so that the stent is flexible in the longitudinal direction; wherein
the plurality of cylindrical elements collectively form first and second stent ends longitudinally separated by a stent body;
at least one of the first and second stent ends is reverse-tapered laterally outward from the longitudinal axis and longitudinally away from the stent body; and
the stent body has a stiffness value of X, and at least one of the first and second stent ends has a stiffness value of Z, with Z being greater than X such that the stent is more resistant to lateral force in the at least one of the first and second stent ends than in the stent body.

2. The stent of claim 1, wherein each of the cylindrical elements includes a plurality of struts interconnected in an angular manner to provide a substantially zigzag aspect to the cylindrical element.

3. The stent of claim 1, wherein both of the first and second stent ends are reverse-tapered outward from the stent body.

4. The stent of claim 1, wherein the stiffness of at least one of the first and second stent ends and the stent body is directly proportional to the length of a plurality of struts in the chosen at least one first and second stent ends and the stent body and also is directly proportional to the strain in the chosen at least one first and second stent ends and the stent body.

5. The stent of claim 1, wherein the ratio of a selected dimension of a plurality of struts forming the at least one reverse-tapered stent end to a corresponding selected dimension of a plurality of struts forming the stent body is 1:A, where A is a chosen number ranging from 1 through 100.

6. The stent of claim 1, wherein at least one cylindrical element forms a transition stent portion longitudinally interposed between a chosen one of the first and second stent ends and the stent body, the transition stent portion has a stiffness value of Y, and Y is greater than X and a chosen one of equal to and less than Z, such that the stent is more resistant to lateral force in the transition stent portion than in the stent body and either the same or less resistant to lateral force in the transition stent portion than in the chosen stent end.

7. The stent of claim 6, wherein the stiffness of at least a chosen one of the first and second stent ends, the transition stent portion, and the stent body is directly proportional to the length of a plurality of struts in the chosen at least one first and second stent end, the transition stent portion, and the stent body and also is directly proportional to the strain in the chosen at least one first and second stent end, the transition stent portion, and the stent body.

8. The stent of claim 6, wherein the ratio of a selected dimension of a plurality of struts forming the chosen stent end to a corresponding selected dimension of a plurality of struts forming the transition stent portion to a corresponding selected dimension of a plurality of struts forming the stent body is 1:A:2A, where A is a chosen number from 1 through 50.

9. The stent of claim 1, wherein the stent defines a stent inner lumen laterally between the cylindrical elements and the longitudinal axis, and wherein the stent includes a flexible lining tissue attached to at least one of the cylindrical elements and substantially lining the stent inner lumen.

10. The stent of claim 2, wherein at least a portion of the plurality of struts are interconnected by at least one intervening bridge beam, at least a chosen one of the struts having a selected dimension, and at least one of the bridge beams having a corresponding selected dimension that is less than the selected dimension of the chosen strut.

11. The stent of claim 1, wherein the stent has a total length between 20 and 200 mm, the stent body has an average expanded diameter between 2 and 50 mm, a plurality of struts forming at least one cylindrical element of at least one of the first and second stent ends are each between 1 and 500 mm long, and a plurality of struts forming at least one cylindrical element of the stent body are each between 1 and 500 mm long.

12. The stent of claim 11, wherein a plurality of struts forming a cylindrical element of a transition stent portion interposed longitudinally between a chosen one of the first and second stent ends and the stent body are each between 1 and 500 mm long.

13. A method of forming a stent having a longitudinal axis extending down a stent inner lumen, the method comprising the steps of:
providing a tubular stent blank laterally enclosing the stent inner lumen and having longitudinally spaced open first and second blank ends separated by a blank body;
reverse-tapering at least one of the first and second blank ends laterally outward from the longitudinal axis and longitudinally outward from the blank body; and
cutting a plurality of apertures in the stent blank to leave behind a plurality of interconnected struts forming the stent, the stent having first and second stent ends longitudinally separated by a stent body; wherein
at least one of the struts is a straight strut, extending substantially parallel to the longitudinal axis, and a plurality of struts forming the at least one reverse-tapered stent end each have a selected dimension that has a predetermined relationship to a corresponding selected dimension of each of a plurality of struts forming the stent body, the predetermined relationship be configured to make the stent is more resistant to lateral force in the at least one reverse-tapered stent end than in the stent body.

14. The method of claim 13, wherein the step of cutting a plurality of apertures in the stent blank includes the step of controlling a laser to cut the plurality of apertures in the stent blank.

15. The method of claim 13, wherein an at least one of the struts is an angled strut, extending parallel to a helix centered about the longitudinal axis.

16. The method of claim 13, wherein a transition stent portion is longitudinally interposed between a chosen one of the first and second stent ends and the stent body, and a plurality of struts forming the transition stent portion each has a selected dimension that has a first predetermined relationship to a corresponding selected dimension of each of a plurality of struts forming the at least one reverse-tapered stent end and has a second predetermined relationship to a corresponding selected dimension of each of a plurality of struts forming the stent body, the first and second predetermined relationships being chosen to make the stent more resistant to lateral force in the transition stent portion than in the stent body and less resistant to lateral force in the transition stent portion than in the at least one reverse-tapered stent end.

17. The method of claim 13, including the step of lining the stent inner lumen with a flexible tissue.

18. A stent, comprising:
a plurality of struts, the struts being interconnected to form a stent having proximal and distal stent ends longitudinally separated by a stent body, a stent inner lumen being laterally enclosed by the proximal and distal stent ends and the stent body, at least one of the proximal and distal stent ends being reverse-tapered laterally outward from the stent body; wherein
at least one of the struts is a straight strut, extending substantially parallel to the longitudinal axis;
at least one of the struts is an angled strut, extending parallel to a helix centered about the longitudinal axis;
a plurality of angled struts are interconnected end-to-end in a zigzag configuration to form a radially expandable cylindrical element extending circumferentially around the stent inner lumen and having a plurality of proximally oriented peaks and distally oriented valleys, each of the proximal and distal stent ends and the stent body being formed by at least one cylindrical element;
a plurality of cylindrical elements being generally aligned along a common longitudinal axis and interconnected by a plurality of interconnecting members with the proximally oriented peaks of one cylindrical element being located laterally inside the distally oriented valleys of an adjacent cylindrical element;
a plurality of substantially longitudinally oriented bridge beams interconnecting adjacent cylindrical elements; and
a plurality of struts forming the at least one reverse-tapered stent end each have a selected dimension that has a predetermined relationship to a corresponding selected dimension of each of a plurality of struts forming the stent body, the predetermined relationship being configured to make the stent more resistant to lateral force in the at least one reverse-tapered stent end than in the stent body.

19. The stent of claim 18, including at least one transition stent portion formed by at least one cylindrical element, the transition stent portion being longitudinally interposed between a chosen one of the proximal and distal stent ends and the stent body, a plurality of struts forming the transition stent portion each having a selected dimension that has a first predetermined relationship to a corresponding selected dimension of each of a plurality of struts forming the at least one reverse-tapered stent end and having a second predetermined relationship to a corresponding selected dimension of each of a plurality of struts forming the stent body, the first and second predetermined relationships being chosen to make the stent more resistant to lateral force in the transition stent portion than in the stent body and less resistant to lateral force in the transition stent portion than in the at least one reverse-tapered stent end.

20. The stent of claim 19, wherein the stiffness of the chosen stent end, the transition stent portion, and the stent body is directly proportional to the length of a plurality of struts in the chosen stent end, the transition stent portion, and the stent body and also is directly proportional to the strain in the chosen stent end, the transition stent portion, and the stent body.

21. A stent, comprising:
a plurality of radially expandable cylindrical elements generally aligned along a common longitudinal axis and interconnected by a plurality of interconnecting members placed so that the stent is flexible in the longitudinal direction; wherein
the plurality of cylindrical elements collectively form first and second stent ends longitudinally separated by a stent body; and
the stent body has a stiffness value of X, and at least one of the first and second stent ends has a stiffness value of Z, with Z being greater than X such that the stent is more resistant to lateral force in the at least one of the first and second stent ends than in the stent body.

22. The stent of claim 21, wherein each of the cylindrical elements includes a plurality of struts interconnected in an angular manner to provide a substantially zigzag aspect to the cylindrical element.

23. The stent of claim 21, wherein at least one of the first and second stent ends is reverse-tapered outward from the stent body.

24. The stent of claim 21, wherein the stiffness of at least one of the first and second stent ends and the stent body is directly proportional to the length of a plurality of struts in the chosen at least one first and second stent ends and the stent body and also is directly proportional to the strain in the chosen at least one first and second stent ends and the stent body.

25. The stent of claim 23, wherein the ratio of a selected dimension of a plurality of struts forming the at least one reverse-tapered stent end to a corresponding selected dimension of a plurality of struts forming the stent body is 1:A, where A is a chosen number ranging from 1 through 100.

26. The stent of claim 21, wherein at least one cylindrical element forms a transition stent portion longitudinally interposed between a chosen one of the first and second stent ends and the stent body, the transition stent portion has a stiffness value of Y, and Y is greater than X and a chosen one of equal to and less than Z, such that the stent is more resistant to lateral force in the transition stent portion than in the stent body and either the same or less resistant to lateral force in the transition stent portion than in the chosen stent end.

27. The stent of claim 26, wherein the stiffness of at least a chosen one of the first and second stent ends, the transition stent portion, and the stent body is directly proportional to the length of a plurality of struts in the chosen at least one first and second stent end, the transition stent portion, and the stent body and also is directly proportional to the strain in the chosen at least one first and second stent end, the transition stent portion, and the stent body.

28. The stent of claim 26, wherein the ratio of a selected dimension of a plurality of struts forming the chosen stent end to a corresponding selected dimension of a plurality of struts forming the transition stent portion to a corresponding selected dimension of a plurality of struts forming the stent body is 1:A:2A, where A is a chosen number from 1 through 50.

29. The stent of claim 21, wherein the stent defines a stent inner lumen laterally between the cylindrical elements and the longitudinal axis, and wherein the stent includes a flexible lining tissue attached to at least one of the cylindrical elements and substantially lining the stent inner lumen.

30. The stent of claim 22, wherein at least a portion of the plurality of struts are interconnected by at least one intervening bridge beam, at least a chosen one of the struts having a selected dimension, and at least one of the bridge beams having a corresponding selected dimension that is less than the selected dimension of the chosen strut.

31. The stent of claim 21, wherein the stent has a total length between 20 and 200 mm, the stent body has an average expanded diameter between 2 and 50 mm, a plurality of struts forming at least one cylindrical element of at least one of the first and second stent ends are each between 1 and 500 mm long, and a plurality of struts forming at least one cylindrical element of the stent body are each between 1 and 500 mm long.

32. The stent of claim 31, wherein a plurality of struts forming a cylindrical element of a transition stent portion interposed longitudinally between a chosen one of the first and second stent ends and the stent body are each between 1 and 500 mm long.
